(19) United States
(12) Patent Application Publication
(10) Pub. No.: US 2023/0233316 A1
(43) Pub. Date: Jul. 27, 2023

(54) HEART VALVE USING VASCULAR GRAFT

(71) Applicant: ACCO Health, Inc., Lakewood, CO (US)

(72) Inventor: Patrick Donohue Rudersdorf, Golden, CO (US)

(21) Appl. No.: 17/582,777

(22) Filed: Jan. 24, 2022

(51) Int. Cl. A61F 2/06; A61F 2/24

(52) U.S. Cl. CPC A61F 2/06; A61F 2/2409; A61F 2220/0075; A61F 2220/005

(57) ABSTRACT

A valve prosthesis assembly is disclosed. The valve prosthesis assembly comprises a replacement valve comprising an attachment cuff and a graft coupled to the attachment cuff of the replacement valve. The graft extends around a portion of the attachment cuff. A size of the graft is configured to be adjusted based on a degree of damage to the artery of the patient.

### BACKGROUND OF THE INVENTION

The present subject matter relates generally to implantable medical devices and methods. Specifically, the present subject matter provides an implantable medical device for the replacement of a pathologic aortic valve and/or aortic root and ascending aorta and a method of implanting the same. The present disclosure further provides an implantable medical device for the repair of an ascending aortic aneurysm, an aortic root aneurysm and/or a sinus of Valsalva aneurysm.

The aortic valve functions as a one-way valve to pump oxygen-rich blood from the left ventricle of the heart into the aorta and then to the rest of the body. Blood is pumped from the left ventricle, through the aortic valve, and into the aorta. Between heart contractions, the aortic valve closes to prevent blood from flowing backward into the heart.

The aortic valve generally includes two or three cusps that, when closed, allow patients to produce a diastolic blood pressure and thus perfusion to all major organs and muscles, including the coronary arteries. The aortic valve leaflets together with the Sinuses of Valsalva and the left main and right coronary arteries form the aortic root. Over time, the valve leaflets or cusps may become stiff and fused, leading to aortic valve stenosis; additionally, certain medical conditions such as rheumatic heart disease or endocarditis may lead to deterioration such as valve destruction, for example. Also, the aortic valve may become incompetent and/or redundant so as to close incompletely, leading to aortic valve regurgitation/insufficiency. In various instances, aortic root aneurysms and aortic dissections can additionally lead to aortic valve regurgitation. Other aortic valve conditions may occur that warrant treatment and/or replacement as well.

Aortic valve replacement is a common heart valve operation wherein an aortic valve of a patient's heart is replaced with an artificial heart valve. During a valve replacement surgery, the surgeon makes an incision in the patient's chest (either by way of a thoracotomy, a median sternotomy, or a hemisternotomy) to provide adequate exposure to the patient's heart. After establishment of cardiopulmonary bypass and cardioplegia arrest, the surgeon then opens the patient's aorta to expose the aortic valve and an incision is made around the annulus of the damaged valve to remove the valve. The surgeon then carefully sews the replacement aortic valve into place and closes the aorta with stitches.

There are two main types of valve prostheses that are used to replace the aortic valve. The first is tissue, which is derived from a bovine (cow), porcine (pig), or cadaveric (human) source. The second type is a mechanical valve prosthesis, which is made from durable materials such as carbon and/or metal, for example. In instances where the replacement valve comprises human tissue, the replacement valve is referred to as a homograft, which is a cadaveric aortic valve harvested from a deceased donor.

Patient Prosthesis Mismatch (PPM) occurs when the normally functioning prosthetic valve does not allow for adequate blood flow through the valve, and results from using a replacement valve that is too small for the size of the patient. PPM results in elevated trans-valvular gradients and is believed to lead to early structural valve deterioration and/or bioprosthetic valve failure. Three techniques known to prevent PPM essentially allow for a larger valve to be implanted at the time of surgery. These techniques include an aortic root replacement (modified Bentall), an aortic annular enlargement, and an aortic root enlargement. Compared to an isolated aortic valve, all of the aforementioned techniques are associated with increased operative technical complexity, increased risk of postoperative complications, increased bleeding risk, and/or increased time on the cardiopulmonary bypass machine. Planning for the future, a larger aortic valve bioprosthesis will enable and/or facilitate transcatheter aortic valve replacements (TAVR) and valve-in-valve TAVR by allowing the placement of a larger valve at the index operation thus decreasing both PPM following the TAVR and the chance of coronary compromise during the operation, for example.

An aortic root replacement is completed using a modified Bentall technique, which includes an aortic root replacement, an aortic valve replacement, and coronary artery re-implantation with a valve-graft conduit. A composite valve-graft includes valve cusps within the aortic root adjacent to a collar, and the collar of the graft is sewn to the annulus of the aortic valve. A Konect aortic valve conduit by Edwards Lifesciences Corp. is an option for use to complete the modified Bentall procedure, for example.

An aortic annular enlargement may be performed to allow for implantation of a larger size valve. Stated another way, an aortic root enlargement enlarges the aortic annulus to allow for a larger prosthetic valve insertion at the time of aortic valve replacement. During such a procedure, the non-coronary Sinus of Valsalva is removed to allow larger valve implantation.

Exemplary techniques for performing an aortic annular enlargement include the Nicks technique, the Ross-Konno technique, and the modified Manouguian technique. The Nicks technique enlarges the root by extending the incision into the aorta across the sinus and to, but not beyond, the annulus. The Ross-Konno procedure enlarges the root by extending the incision from the aortic root into the left ventricular outflow tract. The modified Manouguian technique enlarges the root by extending the incision from the aortic root into the anterior mitral leaflet. In each of these procedures, a wedge-shaped patch is sewn into the partially split leaflet and the ascending aorta. Other techniques exist for performing an aortic annular enlargement as well.

Other common conditions include an aortic root and ascending aortic aneurysm and/or preferential dilation of the non-coronary Sinus of Valsalva (SOV) and an aortic dissection with the tear involving the non-coronary SOV. An aortic root aneurysm is treated by replacing the non-coronary SOV with an aortic valve replacement. The distal extent of the aortic root aneurysm can further involve replacement of the ascending aorta as well if there is an associated aneurysm. An aortic dissection is a tear in the inner layer of the aorta, allowing blood to enter through the tear and cause the inner and middle layers of the aorta to separate. An aortic dissection commonly involves the non-coronary SOV and requires replacement of the non-coronary SOV and replacement of the aortic valve.

Separately, by replacing the non-coronary SOV during an aortic valve replacement surgery would allow the surgeon to implant a larger aortic valve prosthesis, either a bioprosthetic or mechanical aortic valve. Should this valve be a bioprosthetic valve, a larger valve may reduce the chance of patient prosthesis mismatch (PPM) and thus reduce the chance of early structural valve deterioration. Such a bioprosthetic valve would also have the advantage of allowing transcatheter aortic valve replacement (TAVR) at a future date be able to be done with a larger TAVR valve thus reducing TAVR PPM.

In each of the procedures mentioned above, the surgeon typically sews the collar of the replacement valve to the annulus of the aortic valve after the patient's damaged aortic valve is removed.

Accordingly, there is a need for an aortic valve replacement that includes one or more graft portions to allow for easy attachment to the aortic root or aortic annulus. This would allow numerous advantages to the surgeon and patient including, for example, decreased operative times by having a prefabricated valve-graft conduit. The decreased operative times correlate to a decrease in time that the patient is on the heart lung machine and a decrease in time that the patient's heart is arrested, for example. Additionally, such a novel aortic valve replacement having one or more graft portions would allow for ease of treatment of aortic dissections involving an isolated left, right or non-coronary SOV and/or replacement of aortic root aneurysms for an isolated SOV (right, left, or non-coronary SOVs) and/or replacement of ascending aortic aneurysms.

### BRIEF SUMMARY OF THE INVENTION

To meet the needs above and others, an aortic valve prosthesis assembly including a replacement aortic valve comprising an attachment cuff defining an outer circumference is provided herein. The aortic valve prosthesis assembly further comprises a graft coupled to the replacement aortic valve at the attachment cuff. In various instances, the graft is a Dacron® graft, a pericardial graft, or a GORE-TEX® graft. The graft extends around a portion of the outer circumference of the prosthetic valve attachment, or sewing, cuff. The graft is coupled to the replacement aortic valve prior to the aortic valve prosthesis assembly being implanted in a patient. A dimension of the portion of the outer circumference is selectively adjusted and/or tailored based on the degree of damaged aorta that needs to be replaced, for example.

An implantable assembly is disclosed. The implantable assembly comprises a replacement valve comprising an outer circumference, commonly referred to as the sewing cuff. The implantable assembly further comprises a vascular graft coupled/attached to the replacement valve. The graft extends around a portion of the outer circumference, commissure to commissure to allow creation of neoSOVs. The graft is coupled to a mechanical and/or biologic replacement valve prior to the implantable assembly being implanted in a patient. A dimension of the graft is selectively adjusted and/or tailored based on the degree of pathological or damaged aorta that needs to be replaced, for example or to assist in an aortic root enlargement.

In one embodiment, a valve prosthesis assembly for replacement of a valve and a portion of an artery in a patient is provided. The valve prosthesis assembly includes a replacement valve comprising an attachment cuff and a graft coupled to the attachment cuff of the replacement valve. The graft extends around a portion of the attachment cuff. A size of the graft is configured to be adjusted based on a degree of damage to the artery of the patient.

In some embodiments, the replacement valve comprises one of a mechanical valve or a tissue valve. The replacement valve may comprise a plurality of posts annularly extending from the attachment cuff.

In some embodiments, the replacement valve comprises a bottom surface and a top surface, and a main portion of the graft is adjacent the top surface of the replacement valve. A further portion of the graft may be provided adjacent to the bottom surface of the replacement valve.

In other embodiments, the size of the graft comprises a dimension of the portion of the attachment cuff along which the graft is coupled to the replacement valve. In still further embodiments, the size of the graft comprises a surface area of the graft.

In some embodiments, the graft is sewn to the replacement valve. In other embodiments, the graft is coupled to the replacement valve by a biocompatible adhesive.

In another embodiment, a method of replacing a valve and a portion of an artery in a patient is provided. The method comprises the step of providing a valve prosthesis assembly. The valve prosthesis assembly includes a replacement valve comprising an attachment cuff and a graft coupled to the attachment cuff of the replacement valve. The graft extends around a portion of the attachment cuff. The method further includes the steps of adjusting a size of the graft based on a degree of damage to the artery of the patient and suturing the valve prosthesis assembly into the patient.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1A is a perspective view of a valve prosthesis assembly comprising a graft coupled to a mechanical aortic valve prosthesis according to at least one aspect of the present disclosure.

FIG. 1B is a perspective view of a valve prosthesis assembly comprising a graft coupled to a tissue aortic valve prosthesis according to at least one aspect of the present disclosure.

FIG. 2A is a perspective view of the valve prosthesis assembly of FIG. 1A.

FIG. 2B is a perspective view of the valve prosthesis assembly of FIG. 1B.

FIG. 3 is a perspective view of the valve prosthesis assembly of FIG. 1B.

FIG. 4 is an elevational view of the valve prosthesis assembly of FIG. 1A comprising a portion of the graft extending below a bottom surface of the mechanical aortic valve prosthesis.

FIG. 5 is an elevational view of a portion of the valve prosthesis assembly of FIG. 1A.

FIG. 6 is a perspective view of a portion of the valve prosthesis assembly of FIG. 1A.

FIG. 7A is a perspective view of a valve prosthesis assembly utilizing an Edwards Intuity® valve.

FIG. 7B is a perspective view of the valve prosthesis assembly of FIG. 7A.

FIG. 7C is a perspective view of the valve prosthesis assembly of FIG. 7A.

FIG. 8A is a perspective view of a further embodiment of a valve prosthesis assembly including a double layer graft.

FIG. 8B is a perspective view from above of the valve prosthesis assembly of FIG. 8A.

FIG. 8C is a perspective view from below of the valve prosthesis assembly of FIG. 8A.

### DETAILED DESCRIPTION OF THE INVENTION

Human heart valves can become diseased or damaged over time, leading to ongoing conditions such as stenosis or regurgitation. Such pathologies can prevent the heart from working as optimally as it should, thereby requiring a replacement valve. Left untreated, decompensated congestive heart failure can ensue. More specifically, the aortic valve helps keep blood flowing in the correct direction through the heart. The aortic valve is the anatomical junction between a person's heart (i.e. the left ventricle) and a person's systemic circulation (i.e. the aorta). The aortic valve separates the heart's main pumping chamber, or the left ventricle, and the main artery, or the aorta, which supplies oxygen-rich blood to the body. When the aortic valve is not working properly, it can interfere with blood flow and force the heart to work harder to send blood to the rest of the body, which can result in congestive heart failure, for example.

Various congenital conditions exist that cause an abnormal aortic valve formation and thus, function of the aortic valve. The most common such congenital heart condition world-wide is a Bicuspid Aortic Valve (BAV); however, other configurations can exist more rarely (for example, a unicuspid aortic valve or a quadricuspid aortic valve). A certain percentage of individuals with this congenital anomaly also have an associated aortic root and/or ascending aortic aneurysm, which is referred to as BAV Aortopathy.

In various instances, a severe infection, commonly-referred to as endocarditis, can require replacement of the aortic valve and surrounding structures also affected by the infection. At such time, the aortomitral curtain may become infected requiring it to be debrided and replaced, the aortic root may need to be partially debrided, and/or an abscess cavity may need to be debrided. In all such instances, replacement of the aortic valve needs to be occur and major reconstruction of the left ventricle, aortic root, and/or aortomitral curtain is also required.

Severe infections may similarly develop in the pulmonary valve and surrounding structures also affected by the infection. A pulmonary arterioplasty is commonly required at the time of the pulmonary valve replacement and is commonly required for adult congenital heart operations where a revision/reoperation is required.

Aortic dissections commonly affect the functionality of the aortic valve and require, in addition to the aortic valve replacement, one of the sinuses of Valsalva to be replaced along with the ascending aorta.

Aortic valve repair and/or replacement can treat aortic valve disease and help restore normal blood flow while preserving the function of the heart muscle. During an aortic valve replacement, the diseased and/or damaged aortic valve is removed and is replaced with an aortic valve prosthesis, or replacement valve. Replacement valves can be made from one or more materials. In various instances, the aortic valve prosthesis is mechanical and comprised of a durable material such as metal and/or carbon, for example. In other instances, the aortic valve prosthesis is made from cow, pig, and/or human heart tissue. Another type of biological tissue valve replacement that uses a patient's own pulmonary valve is sometimes possible through a Ross procedure, for example; however, any suitable combination of materials that provides a durable, biocompatible valve is envisioned.

In instances of further disease and/or damage to the aortic valve and surrounding structures, a portion or all of the aorta may need replaced. In such instances, the diseased and/or damaged section of the aorta is then removed and replaced with an artificial tube, or graft. Such a graft is typically derived from bovine pericardium or a Dacron® or GORE-TEX® graft is used. During a traditional aortic valve and major root reconstructive surgery, a clinician removes a patient's aortic valve along with the effected section of the aorta. The clinician then sews the graft material and aortic valve into the patient individually. The aortic valve prosthesis assemblies disclosed herein serve to, among other things, streamline the procedure to save invaluable time while also allowing a clinician to personalize the graft to a particular patient's needs. Such a streamlined procedure has numerous advantages including, but not limited to, decreased operative times and/or decreased chance of paravalvular leak. The resultant decreased operative times correlates to decreased cardiopulmonary bypass and/or cross clamp times. The disclosed pre-assembled prosthesis/graft assembly further supports a minimally invasive approach.

As described in greater detail herein, two common conditions require aortic valve replacement. The first is a primary valve problem such as aortic stenosis and/or aortic regurgitation. The second is a primary aorta problem such as an aortic root aneurysm and/or an aortic dissection. Such a primary aorta problem is commonly seen in patients with familial thoracic aorta anomalies, connective tissue disorders, aortic dissections, and/or other idiopathic conditions of the aorta.

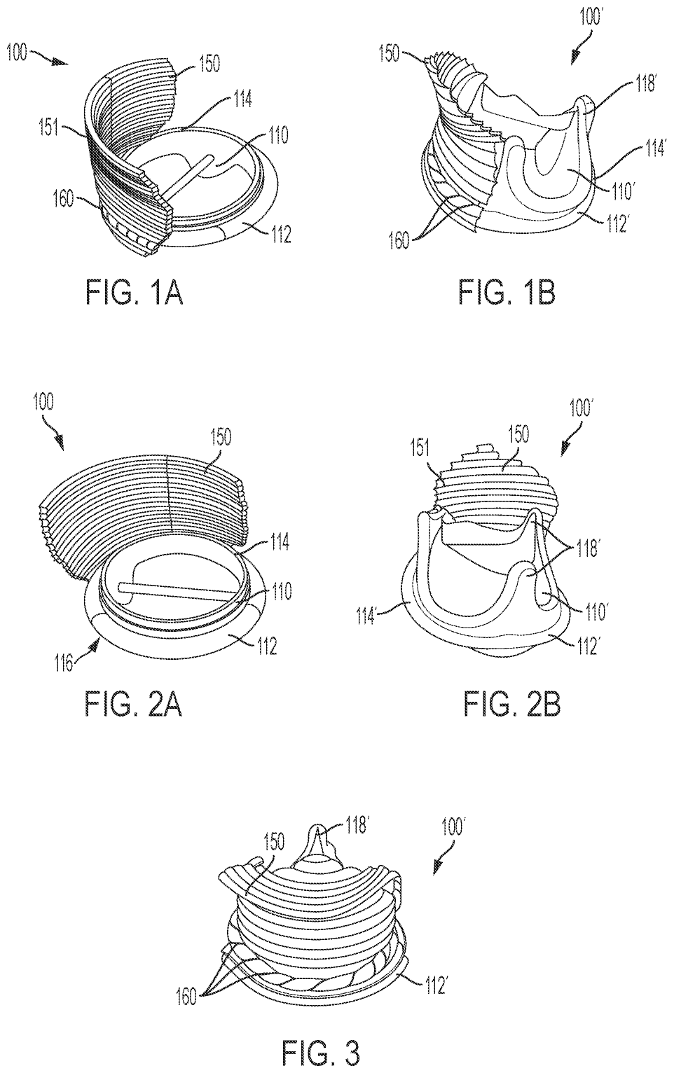

FIGS. 1A and 2A depict a valve prosthesis assembly 100 comprising a mechanical aortic replacement valve 110. As discussed in greater detail herein, the mechanical aortic valve 110 is comprised of any durable biocompatible material, such as carbon and/or metal, for example. The mechanical valve 110 comprises a sewing cuff 112 extending around an outer perimeter of the valve 110 about a central axis. The outer perimeter is defined by an outer circumference of the valve 110. The mechanical valve 110 includes an upper surface 114 and a lower surface 116 opposite of the upper surface 114, the upper and lower surfaces 114, 116 being transverse to the central axis.

FIGS. 1B, 2B, and 3 illustrate a valve prosthesis assembly 100′ comprising a tissue aortic replacement valve 110′. As discussed in greater detail herein, the tissue aortic valve 110′ is comprised of any suitable tissue, such as human, porcine, and/or bovine, for example. The tissue aortic valve 110′ comprises a sewing or attachment cuff 112′ extending around an outer perimeter of the valve 110′. The outer perimeter is defined by an outer circumference of the valve 110′. The tissue aortic valve 110′ comprises a plurality of posts 118′ annularly extending from the sewing cuff 112′. Such posts 118′ mimic commissures naturally present in all aortic valves. Such commissure posts 118′ in prosthetic valves serve to provide support to the prosthetic valve leaflets.

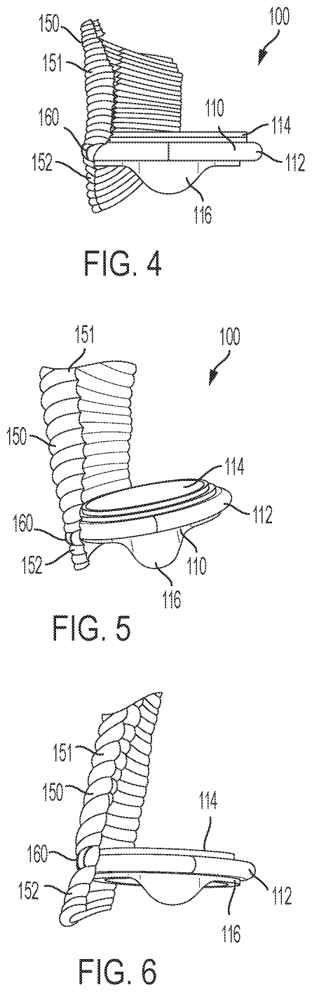

The valve prosthesis assembly 100, 100′ also includes a graft 150 coupled to the valve 110, 110′. The graft 150 can be comprised of any suitable material including, for example, Dacron®, GORE-TEX®, and/or pericardium. The graft 150 extends around a portion of the outer perimeter on the aortic side of the valve 110 and is secured to the valve 110, 110′ at the sewing cuff 112, 112′, respectively. A main portion 151 (see FIG. 4) of the graft 150 adjacent to the aortic side or upper surface 114 of the valve 110 is used for repair or replacement of ascending aortic pathology. In some the embodiment illustrated in FIGS. 1A-3, the graft 150 does not extend toward or beyond the bottom surface 116, 116′ of the valve 110, 110′.

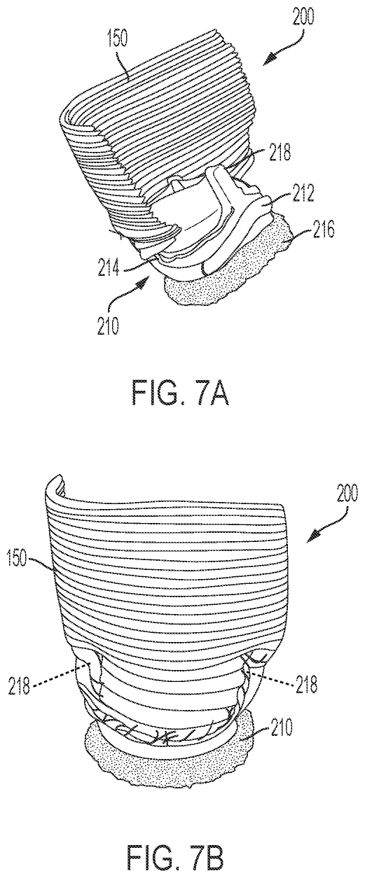

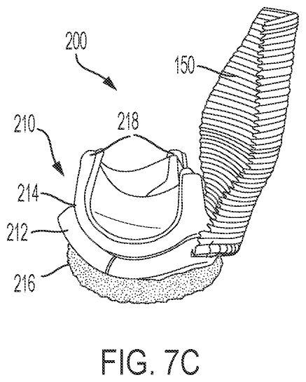

FIGS. 7A-7C illustrate a further embodiment of a valve prosthesis assembly 200 including a graft 150 on an Edwards Intuity® valve 210 by Edwards LifeSciences®. While aortic valves are shown in the illustrated embodiments, other embodiments of the valve prosthesis assembly 100, 100′ include the graft 150 coupled to valves used in pulmonary valve replacement procedures as well.

The graft 150 can be coupled to the valve 100, 100′, 210 in various configurations. For example, as shown in FIGS. 7A-7C, the graft 150 can be secured to the valve 210 around substantially 120 degrees of the circumference of the sewing cuff 212 and along the commissural posts 214, as seen in FIG. 7B. Such attachment has numerous, unique, advantages including decreasing the chance for a paravalvular leak, allowing for concomitant repair of the aortic root along with repair of the ascending aortic aneurysm (possibly in a minimally invasive approach), and/or allowing for use in aortic valve regurgitation (and stenosis) with a large annulus. In other embodiments, the graft 150 is sewn onto the sewing cuff 212 but not along the commissural posts 214, as seen in FIG. 7C.

Alone, the Edwards Intuity® valve 210 is currently only recommended for aortic stenosis patients with smaller annulus. Utilizing a uniquely-fashioned vascular graft 150 with the Edwards Intuity® valve 210 enables concomitant replacement of an aortic valve and/or repair of aortic root/ascending aortic aneurysm in a rapid deployment fashion regardless of valve pathology (i.e. aortic valve regurgitation and/or aortic valve stenosis).

In other instances, the graft 150 can be coupled to the valve 110, 110′, 210 in various configurations to perform a tissue and/or mechanical aortic valve replacement. In a first configuration, the valve prosthesis assembly 100, 100′ is used for addressing aortic valve replacement and repair of the ascending aorta. The graft material 150 is be sewn about 120 degrees along the sewing cuff 112, 112′ of the aortic valve prosthesis 110, 110′ and up along the commissural posts 118′, 218′, allowing for repair of the aortic valve, the aortic root, and/or the ascending aorta.

A second configuration is for performing an aortic root enlargement to decrease patient prosthesis mismatch. In some embodiments, a further portion 152 of the graft material 150 extends below the sewing cuff 112, 112′, for example, to allow the surgeon to attach the graft material 150 to the native aortic valve annulus. In such instances, the graft material 150 is sewn to the aortic valve prosthesis about 120 degrees around the sewing cuff 112, 112′. This allows the surgeon to tailor the graft uniquely intraoperatively. This can also be used to perform an aortoplasty if necessary.

Dimensions of the graft 150 can be customizable based on a number of considerations encountered at the time of surgery of a particular patient. In various instances, the graft 150 extends around a portion of the outer perimeter of the valve 110, 110′. For example, the graft 150 can be sized for use in procedures that require less than a full aortic root replacement. In some embodiments, the graft 150 is secured to approximately 120 to 150 degrees of the outer perimeter or circumference, or the sewing cuff 112, 112′, of the valve 110, 110′. The surgeon may utilize a valve prosthesis assembly 100, 100′ with a relatively larger graft 150 based on interoperative findings of weakened aortic tissue at the time of surgery. The surgeon may also use a valve prosthesis assembly 100, 100′ with a larger graft 150 than originally planned if, during surgery, the surgeon determines that an aortic root enlargement is needed to prevent PPM and/or to allow for the eventual TAVR implantation in younger patients, or to allow for concomitant procedures of the aorta and the valve, and/or to allow for a minimally invasive approach.

The valve prosthesis assembly 100, 100′ includes the graft 150 coupled to the valve 110, 110′, 210 prior to implantation to a patient during a surgical procedure. Such pre-assembly eliminates procedural steps and saves time as compared to self-assembled prosthetic assemblies. Use of the pre-assembled graft 150 further allows for an efficient, time-saving process that can be tailored to the individual, specific needs of a patient. As shown in FIG. 3, the graft 150 is sewn to the valve 110′ around the sewing cuff 112′ using physical stitches 160 of a biocompatible material, such as a non-absorbable suture material, such as silk, nylon, polypropylene, and/or stainless steel, for example. In various instances, the graft 150 is coupled to the valve 110, 110′, 210′ using a biocompatible adhesive, such as glue; however, any suitable bonding material or combination of bonding materials are envisioned that provide a secure coupling between the graft and the valve. During use, the surgeon assesses the amount of damage on the aorta or other artery associated with the valve being replaced and adjusts the size of the graft 150 of the valve prosthesis assembly 100, 100′ to appropriately match the amount of damage prior to inserting valve prosthesis assembly into the patient's body during surgery.

Referring now to FIGS. 4-6, the portion 152 of the graft 150 extends beyond and/or is adjacent to the bottom surface 116 of the valve 110. The extension of the portion 152 of the graft 150 beyond the bottom surface 116 on the ventricular side of the valve 110 allows for sewing into the annulus of a single sinus of Valsalva SOV. The portion 152 of the graft 150 on the ventricular side of the valve 110 can be used for repair or replacement of aortic root pathology and/or aortic root enlargement. In some embodiments, the portion 152 is large enough to allow for replacement of a sinus for aortic root enlargement procedures, replacement of the non-coronary SOV for isolated aortic root aneurysms, or replacement of the non-coronary SOV in the setting of an aortic dissection. During use, the surgeon can assess the amount of damage on the patient's arteries and adjust the size of the portion 152 of the valve prosthesis assembly 100, 100′.

For elective and aortic dissection procedures, the patient-specialized implantable assemblies described herein may be used in an unlimited number of contexts that allow for, without being unduly limited: (1) isolated SOV replacements (e.g. the right-coronary, left-coronary, or non-coronary SOV) in the setting of an AVR; (2) replacement of isolated SOV replacements (e.g. the right-coronary, left-coronary, or non-coronary SOV) and ascending aortoplasty in the setting of an AVR in patients with aortic root and ascending aortic aneurysms; (3) an aortic root enlargement by replacing the non-coronary SOV in the setting of an AVR; (4) an aortic root enlargement by replacing the non-coronary SOV and an aortoplasty in the setting of an AVR to minimize the risk of PPM; (5) the Edwards Intuity® valve to be used in patients with a large aortic annulus and dilated aortic root with replacement of a single SOV (right, left, or non); and/or (6) minimally invasive applications for surgery of the aortic valve, aortic root, and ascending aorta.

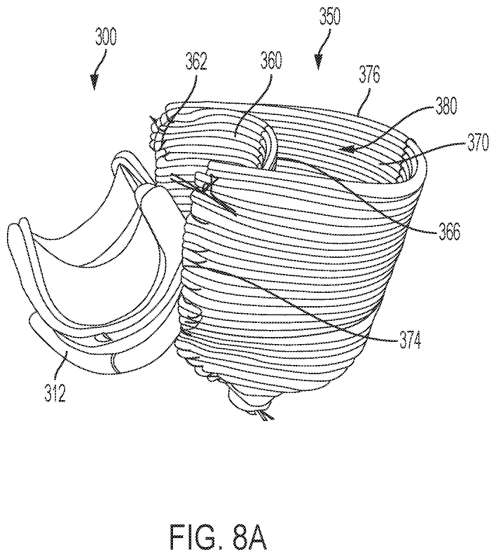

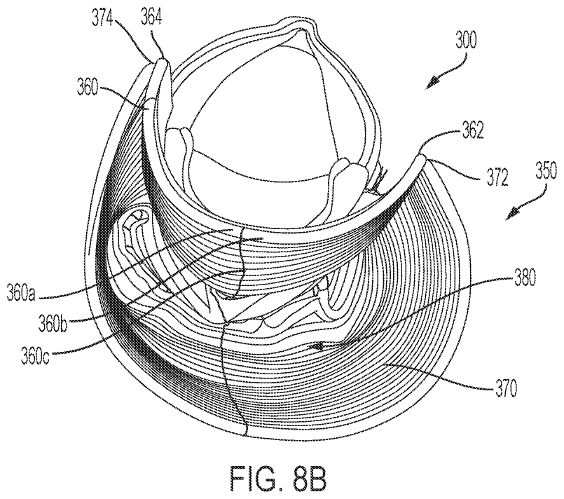

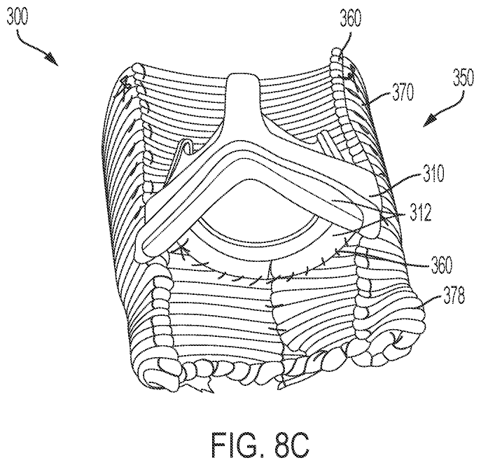

Referring now to FIGS. 8A-8C, an additional embodiment of a valve prosthesis assembly 300 is depicted. The valve prosthesis assembly 300 is similar in many respects to the valve prosthesis assemblies 100, 100′ shown in FIGS. 1A and 1B, respectively. In the illustrated embodiment, the valve prosthesis assembly 300 includes an aortic replacement valve 310. In various instances, the aortic replacement valve 310 is mechanical and is comprised of any durable biocompatible material, such as carbon and/or metal, for example. In other instances, the aortic replacement valve 310 is a tissue aortic valve and is comprised of any suitable tissue, such as human, porcine, and/or bovine, for example.

The aortic replacement valve 310 comprises a sewing cuff 312 extending around an outer perimeter of the valve 310, which is defined by an outer circumference of the valve 310. The aortic replacement valve 310 further comprises a graft 350 coupled thereto, which is similar in many respects to graft 150. The graft 350 can be comprised of any suitable material including, for example, Dacron®, GORE-TEX®, and/or pericardium. The graft 1050 is designed to extend around a portion of the outer perimeter on the aortic side of the valve 310 and is secured to the valve 310 at the sewing cuff 312. The graft 350 on the aortic side of the valve 310 can be used for repair or replacement of ascending aortic pathology, and the graft 350 is configured to be coupled to the sewing cuff 312 of the aortic replacement valve 310 through any suitable attachment mechanism 360, including sutures, for example.

In the embodiment provided in FIGS. 8A-8C, the graft 350 includes two layers 360, 370 positioned side by side. Stated another way, the graft 350 is double-layered. A first, innermost layer 360 is configured to be positioned adjacent the valve 310 and includes a first commissure 362 and a second commissure 364. The first graft layer 360 is imbricated, i.e., includes overlapping edges. For example, the first graft layer 360 includes a first portion 360a and a second portion 360b that overlap at internal edges and are stitched together along a stitching line 360c. A second, outermost layer 370 is configured to form an exterior surface of the valve prosthesis assembly 300. Similarly, the second graft layer 370 comprises a first commissure 372 and a second commissure 374. In some embodiments, the second graft layer 370 may also be imbricated.

The first graft layer 360 is configured to be coupled to the second graft layer 370 by way of sutures, for example; however, any suitable attachment mechanism is envisioned. In such instances, the first graft layer 360 and the second graft layer 370 are sutured together along the first commissures 362, 372 and the second commissures 364, 374. The first graft layer 360 and the second graft layer 370 are coupled together on the ventricular side along the bottom surface 316 of the sewing cuff 312 of the replacement valve 310. A distal end 366 of the first graft layer 360 is sewn, or otherwise coupled, to a distal end 376 of the second graft layer 370. When secured within the body, the distal ends 366, 376 of the graft layers 360, 370 are coupled together into the distal anastomosis of the aorta on a second side of the replacement valve 310 opposite the ventricular side. Proximally, the graft layers 360, 370 are coupled together and to the aortic valve annulus. Such coupling of the first graft layer 360 and the second graft layer 370 creates and/or defines a space or chamber 380 between the two layers. As discussed in greater detail herein, the defined space 380 allows for a replacement valve to be inserted therein as the aortic valve 310 deteriorates and/or malfunctions.

The illustrated embodiment of the valve prosthesis assembly 300 also includes a portion 378 of the outer, second graft layer 370 that extends beyond the sewing cuff 312 below the bottom surface 316 of the valve 310. The portion 378 on the ventricular side of the valve 310 allows for sewing into the annulus of a single sinus of Valsalva SOV.

Such a novel double-layered graft 350 provides numerous benefits in areas including long-term patient care. For example, over time, tissue and/or bioprosthetic heart valves degenerate and eventually fail, requiring the need for replacement. Transcatheter aortic valve replacement (TAVR) technology offers a minimally invasive procedure to patients who previously underwent open-heart surgery for a heart valve replacement. TAVR is a minimally invasive procedure where a new valve is inserted without removing the old and/or damaged valve. Using the less invasive “valve-in-valve” procedure, a new valve is placed into an orifice of the failed surgical valve to relieve any valve dysfunction. The TAVR approach delivers a collapsible replacement valve to the valve site through a catheter, for example, the new valve expanding and pushing the old valve leaflets out of the way and the tissue in the replacement valve takes over the job of regulating blood flow.

The space or chamber 380 defined between the first and second graft layers 360, 370 allows for expansion during a valve-in-valve TAVR procedure. This expansion allows for the minimally invasive placement of a larger TAVR valve. Furthermore, as discussed above, the first, innermost graft layer 360 is imbricated. This overlap of graft material allows for an increased area for a valve-in-valve TAVR. When the surgical valve 310 is cracked and/or broken at the time of the valve-in-valve TAVR, the first graft layer 360 will also “crack,” thereby separating the overlapped material and allowing for the placement of a large valve-in-valve TAVR.

In various instances, the attachment mechanisms used throughout this disclosure to couple the graft(s) to a sewing cuff of a valve and/or to couple first and second graft layers to one another are flush with the material of the graft. In other instances, the attachment mechanism, such as sutures, extend from and/or lay on top of an exterior surface the graft material.

The implantable assemblies described herein are envisioned for use in the replacement and/or repair of valves other than the aortic valve. For example, the implantable assemblies can be used for treatment of the mitral valve for severe mitral annular calcification where a patch repair of the mitral valve annulus is required after debridement. Furthermore, the implantable assemblies described herein can be used in the treatment of endocarditis when the aortomitral curtain or a portion of the left atrium needs to be replaced and/or repaired for an infectious etiology.

It should be noted that various changes and modifications to the presently preferred embodiments described herein will be apparent to those skilled in the art. Such changes and modifications may be made without departing from the spirit and scope of the present invention and without diminishing its attendant advantages.

What is claimed is:

1. A valve prosthesis assembly for replacement of a valve and a portion of an artery in a patient, the valve prosthesis assembly comprising:
a replacement valve comprising an attachment cuff; and
a graft coupled to the attachment cuff of the replacement valve, wherein the graft extends around a portion of the attachment cuff;
wherein a size of the graft is configured to be adjusted based on a degree of damage to the artery of the patient.

2. The valve prosthesis assembly of claim 1, wherein the replacement valve comprises one of a mechanical valve or a tissue valve.

3. The valve prosthesis assembly of claim 1, wherein the size of the graft comprises a dimension of the portion of the attachment cuff along which the graft is coupled to the replacement valve.

4. The valve prosthesis assembly of claim 1, wherein the graft is sewn to the replacement valve.

5. The valve prosthesis assembly of claim 1, wherein the graft is coupled to the replacement valve by a biocompatible adhesive.

6. The valve prosthesis assembly of claim 1, wherein the replacement valve comprises a bottom surface and a top surface, and a main portion of the graft is adjacent the top surface of the replacement valve.

7. The valve prosthesis assembly of claim 6, wherein a further portion of the graft is adjacent the bottom surface of the replacement valve.

8. The valve prosthesis assembly of claim 1, wherein the replacement valve comprises a plurality of posts annularly extending from the attachment cuff.

9. A method of replacing a valve and a portion of an artery in a patient, the method comprising:
providing a valve prosthesis assembly comprising:
a replacement valve comprising an attachment cuff; and
a graft coupled to the attachment cuff of the replacement valve, wherein the graft extends around a portion of the attachment cuff;
adjusting a size of the graft based on a degree of damage to the artery of the patient;
suturing the valve prosthesis assembly into the patient.

10. The method of claim 9, wherein the replacement valve comprises one of a mechanical valve or a tissue valve.

11. The method of claim 9, wherein the graft is sewn to the replacement valve.

12. The method of claim 9, wherein the graft is coupled to the replacement valve by a biocompatible adhesive.

13. The method of claim 9, wherein the replacement valve comprises a bottom surface and a top surface, and a main portion of the graft is adjacent the top surface of the replacement valve.

14. The method of claim 13, wherein a further portion of the graft is adjacent the bottom surface of the replacement valve.
